(19) United States
(12) Patent Application Publication
(10) Pub. No.: US 2015/0118313 A1
(43) Pub. Date: Apr. 30, 2015

(54) ENHANCED FOLIC ACID FLUORESCENT MATERIAL, MULTIFLUORESCENT POROUS COMPOSITIONS OF MATTER AND POTENTIAL APPLICATIONS THEREOF

(71) Applicant: Nanologica AB, Stockholm (SE)

(72) Inventors: Alfonso E. Garcia-Bennett, Stockholm (SE); Chunfang Zhou, Stockholm (SE)

(21) Appl. No.: 14/585,451

(22) Filed: Dec. 30, 2014

Related U.S. Application Data
(62) Division of application No. 13/635,098, filed on Nov. 16, 2012, now abandoned.
(60) Provisional application No. 61/364,424, filed on Jul. 15, 2010.
(60) Provisional application No. 61/314,809, filed on Mar. 17, 2010.

(51) Int. Cl. C09B 63/00; A61K 47/22; A61K 41/00; A61K 33/24; A61K 9/14

(52) U.S. Cl. CPC C09B 63/00; A61K 33/24; A61K 9/145; A61K 41/0057; A61K 9/143; A61K 47/22
USPC 424/489; 424/649; 435/7.21

(57) ABSTRACT

A method for the preparation of enhanced fluorescent folic acid mesoporous material, multifluorescent mesoporous materials, their novel properties and applications such as: a mesoporous fluorescent composition suitable for printing identification marks on metals, glass, plastic, ceramics, or paper which are visible only when excited by an external radiation; and applications in life science applications such as diagnostic, biodistribution markers, and targeted drug delivery applications.

### RELATED APPLICATIONS

This application is a Divisional of U.S. application Ser. No. 13/635,098 filed on Sep. 14, 2012, which is a National Phase filing under 35 U.S.C. §371 of PCT/EP2011/054009 filed on Mar. 16, 2011; and this application claims the benefit of U.S. Provisional Application No. 61/314,809 filed on Mar. 17, 2010, and U.S. Provisional Application No. 61/364,424 filed on Jul. 15, 2010. The entire contents of each of these applications is hereby incorporated by reference.

### DESCRIPTION

1. Technical Field and Description of the Invention

This present invention is directed towards a method for the preparation of enhanced fluorescent folic acid mesoporous material, multifluorescent mesoporous materials, their novel properties and applications such as: a mesoporous fluorescent composition suitable for printing identification marks on metals, glass, plastic, ceramics, or paper which are visible only when excited by an external radiation; and applications in life science applications such as diagnostic, biodistribution markers, and targeted drug delivery applications. The present invention encompasses additionally the formation of a nanoporous reactor vessel for activated self-assembled units of folic acid plus one or more active compounds through the formation of said fluorescent material.

More specifically the invention provides a fluorescent mesoporous particle, comprising folic acid tetramers plus one or more active functional compounds which may include; fluorescent molecules, pharmaceutical active drugs, and others limited only by their availability to form certain type of interactions such as π-π and π-σ stacking with folic acid.

Compositions of matter self-assembled using methods in the embodiment offer enhanced properties in comparison to the un-assembled components. For example, the fluorescent properties of folic acid or folic acid plus porphyrin are enhanced in intensity when self-assembled in order to form mesoporous silica materials in comparison to the single components not self-assembled as described in the embodiment, or loaded post-synthetically into mesoporous silica.

Folic acid plus pharmaceutical drugs provide materials capable of targeting to specific cells, in particular tumor cells, and delivering pay loads of pharmaceutical drugs in a controlled delivery system. This may provide useful early cancer diagnosis tools, as well as targeting and therapeutic strategies for cancer treatment. The invention also provides a photosensitize carrier for the photodynamic treatment. The invention hence provides imaging, targeting, diagnosis, and therapy functionalities into one porous material product. Hence, the invention here described provides for the formation of theranostics materials.

2. Background Art

Since the first reported preparation of silica-based material MCM-41 [Kresge C. T.; et al. Nature, 1992], mesoporous solids have attracted much attention in various research fields due to their potential in diverse application areas ranging from catalysis to biomimetic engineering, sensor technology, bio imaging of cancer cells and drug delivery. These materials have been studied in depth from synthetic, structural and applied perspectives. Recently, Che and co-workers reported the preparation of highly ordered mesoporous structures AMS-n using anionic surfactants and co-structure-directing agents (CSDAs) [Che S.; et al. Nature Materials, 2003]. New mesoporous materials NFM-1 utilizing pterin containing molecules such as folic acid as pore forming agents or templates were developed and are described in WO/2009/068117. Commercially interesting and relevant advantages of these preparation routes are the ability to incorporate a variety of organic functional groups in one synthetic step as the CSDA, which remain covalently bound within the internal pore surface of the mesoporous material after extraction of the surfactant. [Garcia-Bennett A. E.; et al. Angew. Chemie Int. Ed., 2005]. The use of non-surfactant templates such as folates offers further additional advantages; as these are non-toxic compounds (folic acid is a member of the vitamin B group of molecules); folates are chiral molecules and impart chirality to the covalently bound functional groups; folic acid interacts via π-π and π-σ stacking with a large variety of biologically and electronically active molecules, and hence allows them to be incorporated within the internal pore space of the mesoporous solid formed. [Garcia-Bennett A. E.; et al, J. Am. Chem. Soc. 131(9) 3189-3191, 2009]

Within molecular diagnostic and bioimaging applications, multifunctional devices capable of providing on the one hand; sensitive conjugation to proteins, enzymes or antibodies at low concentrations; and secondly strong detection signals in fluorescence mode are highly desirable. Furthermore, a major goal in the preparation of such devices is to provide multiple fluorescent signals within the single detection device for such applications as flow cytometry. In order to achieve this, fluorophores with large Stokes shifts are required, which would allow to separate otherwise merging emission peaks when a variety of fluorophores are combined. Porphyrin is an important class of natural and artificial pigments with the large Stokes shift. However, the problem is that many of the physicochemical properties of this class of pigments, and in particular the electronic absorption and the luminescence properties, are strictly dependent on their aggregation behavior. For example, their aggregation will decrease the luminescence efficiency and photo-oxidation efficiency within in photodynamic therapy (PDT) applications. The encapsulation of fluorophores and dyes and/or pigments within ordered mesoporous materials offers the potential to control their aggregation behavior whilst retaining the internal and external surface of the mesoporous particle for further conjugation with biomolecules (DNA, proteins, enzymes, etc.), analytes (cations, anions, etc.) and other relevant molecules including cellular targeting agents.

Meso-tetrakis (4-carboxyphenyl) porphyrin (TCPP) is being exploited as a marker for the rapid detection of tumor cells by fluorescence imaging. [Li, Wen-Tyng, Current Drug Metabolism, 10(8), 851-860, 2009] TCPP possess a large stokes shift of 260 nm having excitation and emission peak wavelengths of approximately 414 nm and 674 nm respectively. Folic acid molecules have a very weak natural fluorescent intensity . They are composed of a pterin group a benzoic acid group and a chiral glutamic acid moiety. They are excellent examples of molecules that may self-assemble with folic acid forming hexagonal liquid crystal assemblies capable of acting as pore forming agents for the preparation of mesoporous materials with new functions.

TCPP is a weak acid, and as such it is easily neutralized in the range of pH 5-7 (pKa 6.6), leading to a neutral species which is scarcely soluble in aqueous solutions. At pH<3.5 the nitrogen atoms of the TCPP core are protonated, yielding a dicationic porphyrin which is able to self-assemble into J-aggregates. [Clarke, S. E.; J. Phys. Chem. A, 2002] At higher pH value, TCPP will loses four protons of carboxyl groups and form H₂TCPP⁴⁻ with negative charges. In the present preparation method we have chosen Borax-Boric acid buffer with pH=9.2 as solvent, in order to ensure H₂TCPP⁴⁻ molecules which can interact with the protonated aminopropyl group of NFM-1. Structures of meso-tetrakis(4-carboxyphenyl)porphyrin and its ionic species are shown in the following Scheme 1.

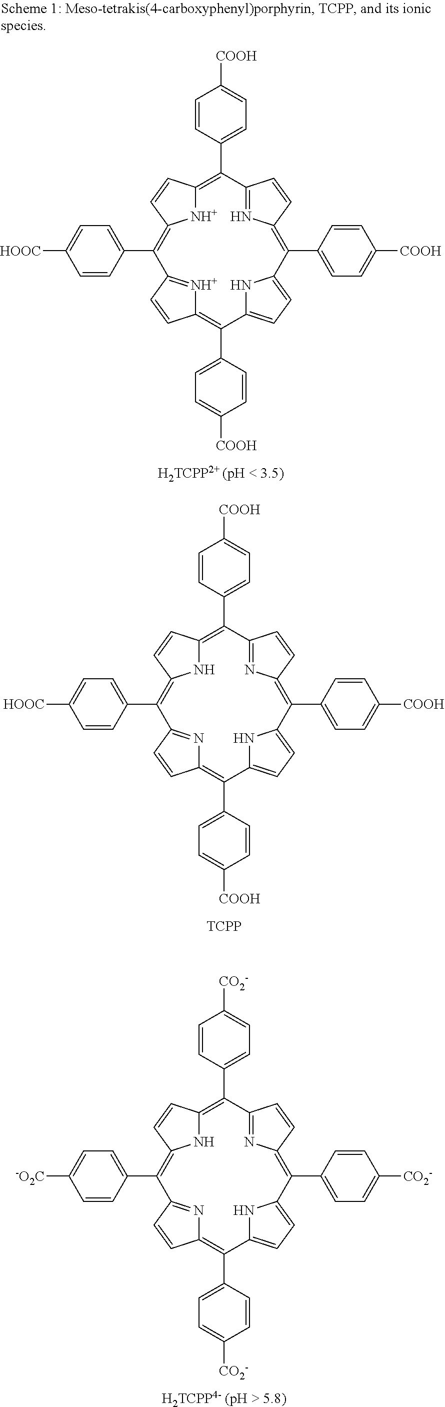

When such materials are prepared the optical characteristics will differ considerably due to the formation of the folate-porphyrin aggregates, their local environment and their inclusion (confinement) within an ordered inorganic oxide based mesoporous matrix. For example changes in fluorescence intensity and shifts towards higher and lower emission wavelengths are expected. These are based on the Förster resonance energy transfer, FRET, properties of donor and acceptor aggregates.

Hence another characteristic of the embodiment is the possibility to produce novel efficient multifluorescent materials, with high porosity and groups capable of binding to biologically relevant molecules. Multifluoresence occurs when more than one optically active fluorescent molecule are encapsulated together, without their emission bands merging together. Multifluorescence labels or stainers are useful in applications in confocal microscopy and immunology assays, amongst others. A multifluoresence material can be prepared using the embodiment of the invention here described using porphyrin and folate fluorescent molecules; however other optically active molecules may be used. Fluorescein isothiocyanate (FITC) is a derivative of fluorescein used in wide-ranging applications including flow cytometry. FITC has an excitation and emission spectrum peak with wavelengths of approximately 495 nm/521 nm respectively which does not overlap with the emission and excitation bands of porphyrin nor folic acid and hence an ordered mesoporous material with three separated emission bands is described in this invention. The following Table 1 summarizes the different wavelengths of excitation and emission of the free molecules referred to in the aforementioned text, and included here as examples of suitable optically active fluorescent molecules for the purpose of this invention.

TABLE 1

| Fluorescent Name | Excitation Max. (nm) | Emission Max. (nm) |
|---|---|---|
| Folic acid | 280 & 368 | 450 |
| FITC | 500 | 515 |
| TCPP | 414 | 670 |

The present invention also provides an assay method for tracking the movement of cells or a cellular component, or biodistribution in vitro and in vivo. Suitable means for detecting, recording, measuring, or imaging in the embodiment of the invention are known in the art and include, for example, a flow cytometer microscope, a confocal microscope, a laser scanning cytometer, a fluorescence micro-plate reader, a fluorescent microscope, a bright-field microscope, a high content scanning system, and like device.

The multifunctional particles with folic acid (for example Folic acid and porphyrin, folic acid and antifolates, or folic acid plus cis-Pt) may be used as targeting agents for the delivery of active pharmaceutical products to tumor cells (for example cis-Pt, porphyrin or antifolates), since these have been shown to express higher concentrations of folate receptors on their membrane surfaces. This is particularly true for certain variations of cancer (e.g. pulmonary and ovarian cancer cell line). The multifunctional particles prepared under the scope of this invention can directly deliver the therapeutic agents to a desired location with a variety of clathrin and non-clathrin based uptake mechanisms. [Garcia-Bennett A. E., et al. Biochem Pharmacol. In press 2011 Feb. 12.]

### BRIEF DESCRIPTION OF THE DRAWINGS

FIG. 1a shows release kinetics curves of folic acid in NFM-1-F (fiber), NFM-1-R (rods), NFM-G (gyroids) and NFM-1-X (amorphous) particles;

FIG. 1b shows fluorescence emission spectra of the released folic acid from the nanoporous particles with the different morphologies with λex=288 nm;

FIG. 2 shows XRD patterns at low and high angle: a) NFM-1, b) NFM-1P(367), c) NFM-1P(16), d) NFM-1P(8),e) NFM-1P(5);

FIG. 3 shows fluorescence emission spectrum of the solution released from NFM-1P(x) particles compared to free folate and folate and TCPP in solution. λex=368 nm. Note: NFM-1P(x) actually denoted NFTCPP-1(x) on figure;

FIG. 4 shows release kinetic curves of folic acid and TCPP in NFM-1P samples;

FIG. 5 shows XRD patterns of NFM-1 with the different amount of cisplatin at low and high angle;

FIG. 6 shows fluorescence emission spectrum of the folic acid solution released from NFCP-1 (x) particles compared to free folic acid and cis-platin solution with the same folic acid concentration of 0.078mg/l. λex=288 nm;

FIG. 7 shows kinetic release profile of cis-platin from 150 mg NFCP-1 (2) in 750 mL PBS buffer at 37 oC under 150 rpm stirring;

FIG. 8 shows kinetic release profile of Folic acid from 150mg NFCP-1 (2) in 750 mL PBS buffer at 37 oC under 150 rpm stirring;

FIG. 9 shows a scheme of TCPP molecules stacked into the hexagonal structure of folic acid in solution and in the final solid;

FIG. 10 shows a scheme of potential methods for using materials according to the invention.

### DETAILED DESCRIPTION OF THE INVENTION

The present invention includes a simple method to produce single fluorescent or multiple fluorescent mesoporous particles whereby the particles may possess porosity. The invention is applicable for the preparation of ordered and disordered mesoporous materials, together with fluorophores, pharmaceutical active compounds, vitamins and flavors; or compounds capable of forming π-π and π-σ stacking interactions. However, the invention is more suited to the synthesis of ordered mesoporous materials prepared directly with folic acid and related folate derivatives as pore forming agents as these are fluorescent themselves and considerably enhance in fluorescence once they are loaded into mesoporous silica materials as described below, in comparison to free folates in solution or the same loaded into mesoporous materials via post-synthetic methods.

The invention demonstrates that several fluorophores (used here to represent an active compound capable of forming π-π and π-σ stacking interactions) may also be incorporated directly in the synthesis. Examples of flurophores suitable for the present invention include: Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, TRITC, X-Rhodamine, Lissamine Rhodamine B, Texas Red, Allophycocyanin (APC), APC-Cy7 conjugates.

This embodiment of this invention may be used for photodynamic therapy (PDT). PDT is a two-step treatment process which has been found to be effective in destroying a wide variety of cancers cells. [Huang et al., Technol Cancer Res Treat. 2005 June; 4(3): 283-293]. PDT is performed by first systemically or topically administering a photosensitize compounds, and subsequently illuminating a treatment with the light in a waveband, which activates the photosensitize compound, causing it to destroy the diseased tissue, [see for examples: U.S. Pat. No. 6,210,425 and U.S. Pat. No. 6,454,789].

Examples of the photosensitize agents for the purpose of this invention can be clinical photosensitizers such as Temoporfin, Porfimer sodium, Vertiporfin, lutexaphyrin, Talaporfin, HPPH, Phthalocyanine.

The following preparation steps are employed in preparing and evaluating multifunctional fluorescent mesoporous particles based on π-π and π-σ stacking interactions and using porphyrin as an example:

### Step A

This invention refers to the formation of mesoporous particles known as NFM-1 as described in patent WO/2009/068117 previously. Step A involves an addition to this method as described in said patent whereby a therapeutic agent capable of interacting with folic acid. The fluorescent or therapeutic agents should be capable of forming interactions such as π-π or π-σ or other stacking interactions or interactions involving delocalized electrons as those found in conjugated groups. Step A involves adding the desired amount of the therapeutic agents (such as cisplatin, porphyrin derivatives, atorvastatin, simvastatin, methotrexate or mixtures) to a solution containing folic acid in an aqueous solvent such as water, other polar solvents such as alcohols, or non-polar solvents (toluene, benzene etc.) or mixtures of the above. The best mode in the case of TCPP is achieved using an aqueous solution, despite both folic acid (FA) and TCPP having poor solubility in this solvent. The solubility of TCPP and folic acid in water is dependent on pH. Aqueous solubility of the therapeutic compound or compounds is not a pre-requisite for the successful completion of step A.

The ratio of FA:additional fluorescent or therapeutic compound may be varied from 1:500 to 1:1 but is particularly interesting in the range between 1:20 and 1:1, as the final material will possess optimum fluorescent properties, as described in the examples below. Ordered mesoporous materials are not formed at ratios of 1:1 as for the TCPP incorporation.

The molar ratio of template molecule to water or the other solvents as exemplified by the use of folic acid, (FA:H₂O), can be varied from 0.1:1 to 0.001:1, but better structural order is achieved in the range between 0.0015:1 and 0.003:1. A common practice is to perform Step A in the presence of buffer solutions containing different degrees of salts, for examples phosphate buffer saline solution. The use of this depends on the fluorescent or therapeutic agent chosen to be loaded.

### Step B

Step B involves adding a chemical substance or substances to the solution under stirring or ultrasonic treating. The chemical substance may also promote or affect the formation of Hoogsteen-type interactions between pterin or similar groups within the folic acid through a variation of pH (see diagram 1). This chemical substance is typically composed of a basic group such as an amine moiety, bonded to an alkyl spacer which may vary in length (propyl, butyl, pentyl, etc) which is in turn bonded to a alkoxy silane. An example of such a molecule is aminopropyl triethoxy silane, APES. The ratio APES:FA may vary from 0.02:1 to 3:1, whilst an optimum material is achieved with ratios varying between 0.2:1 and 2:1. The mixture is stirred or ultrasonic treated at a temperature between 4° C.-100° C. that allow the substances to be homogeneous mixed under an appropriate amount of time.

The increase in pH caused by addition of groups such as APES, causes in addition the solubility of both the TCPP and FA molecules to increase and fully dissolve in the resulting solution, which may have a pH of between 6-10.5, but preferably between 7-9.5.

### Step C

Mixing the solution with at least one metal oxide precursor. Suitable metal oxide precursors may be formed from any oxide of; silica, alumina, titanium, nickel, copper, cobalt, iron, indium, tin, nickel, ruthenium and rhodium, and/or mixtures of the above. The silicon alkoxide Tetraethyl orthosilicate, (TEOS) is especially preferred in this case. If TEOS is used in this step the TEOS:H₂O ratio is preferable between 1:100 and 1:400. The TEOS is added to the solution under vigorous stirring at a temperature which may vary between 4° C.-100° C. and kept in those conditions for at least 10 min, in order to homogenize it.

### Step D

Solidifying the mixture through the sol-gel transition. The conditions have to be chosen so as to induce the sol-gel transition of the reacting solution. This can be done by controlling the amount of thermal energy per gram solution and per unit time which are applied to the reacting solution until the sol gel transition occurs. The amount of energy applied to the solution during the first three hours is preferable between 0.1 and 10 Joule per minute and gram solution, preferable between 0.5 and 3 Joule per minute and gram solution. This can be done by keeping the solution in an appropriate sealed vessel at a temperature between 20 and 120° C. preferable between 40 and 80° C., for at least 6 hours, but maybe as long as 10 days. The temperature has to be chosen according to the thermal conductivity of the vessel and the amount of reacting solution. If the vessel and solution have a lower temperature than the surrounding, heat from the surrounding is transferred to the vessel and solution by conduction. A hydrothermal treatment may also be necessary to promote condensation. This is conducted at 80° C. for a period of between 5 hours and 5 days. The length of time of the hydrothermal step may be decrease if a higher temperature is used. The material may be filtered using conventional filtering methods utilizing filter paper.

### Step E

This step concerns a method to release or partly release the stacks of folic acid and therapeutic agents. The release process could be finished in the temperature of 4° C.-100° C. The optimum temperature is in the range of 25° C.-40° C. The release solvent can be water, buffer or organic solvent. The best one in the invention is the buffer with pH value of 7.4. As for the partly released samples, it could release 5%-100% of the folic acid dependent on the release solvent and time. If 30% of folic acid are released from the nanoporous particles, about 70 m²/g surface area can be obtained.

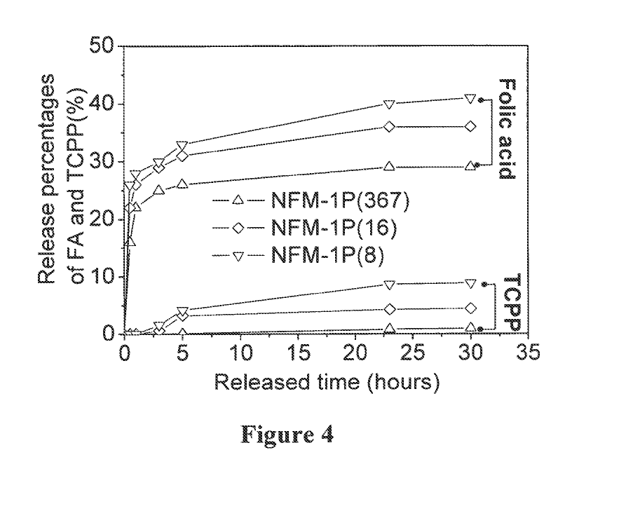

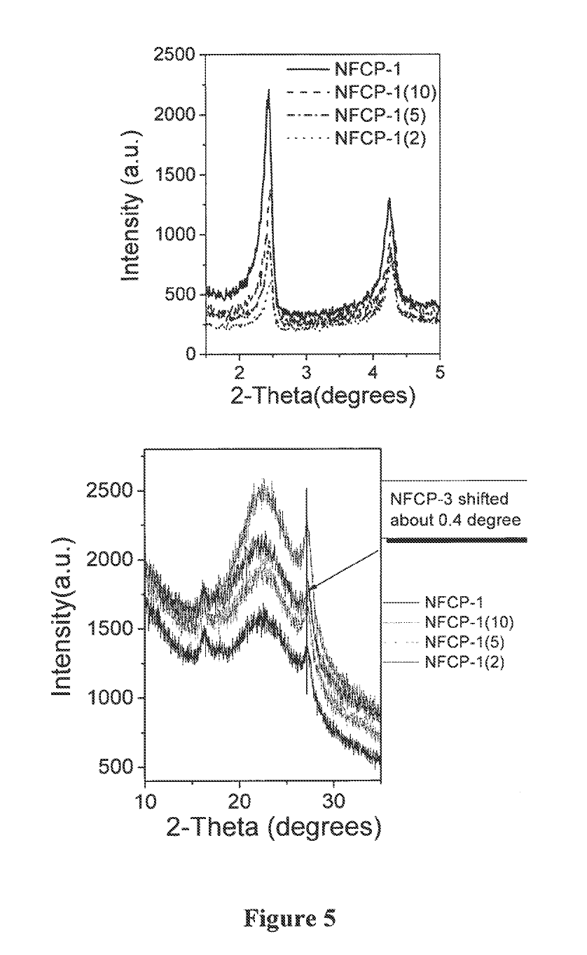

FIG. 4 shows a release curve for folic acid and TCPP from the internal pore space of an NFM-1 particle. The kinetic release curves demonstrate that TCPP and Folic acid tetramers are released at the same rate indicating that these are release as stacks of alternating folate tetramers and TCPP molecules. The formation of folate+TCPP stacks is inferred by the enhanced fluorescence intensity obtained from solutions containing the released TCPP and folate tetramer molecules.

### Step F

The invention includes a step whereby the materials may be functionalized with organic groups on the mesoporous surface. This can be performed through post-grafting methods or through direct grafting methods for both examples of the material synthesis routes used (direct synthesis and post-synthesis). Examples of typical functional groups that can be attached include; amine groups R—NH₂, carboxylic acid groups R—COOH, thiol groups R—SH, cyano groups R—CN, etc. where R is typically an alkane chain

### Step G

The particles in the invention may be formulated for parenteral administration (e.g., by injection, for example, bolus injection or continuous infusion) and may be presented in unit dosage form in ampules, pre-filled syringes, small volume infusion containers or multi-dose containers with an added preservative, or for formulation in aerosols. The pharmaceutical compositions may be composed of the NFM-1 particles with the fluorescent or therapeutic agents as suspensions, solutions, or emulsions of in oily or aqueous solvents and may be the folate stacks with the therapeutic agents which were released from the nanoporous particles. Alternatively, the pharmaceutical compositions of the invention may be in powder form, obtained with a suitable vehicle before use.

### EXAMPLES AND DIAGRAMS

### Example 1

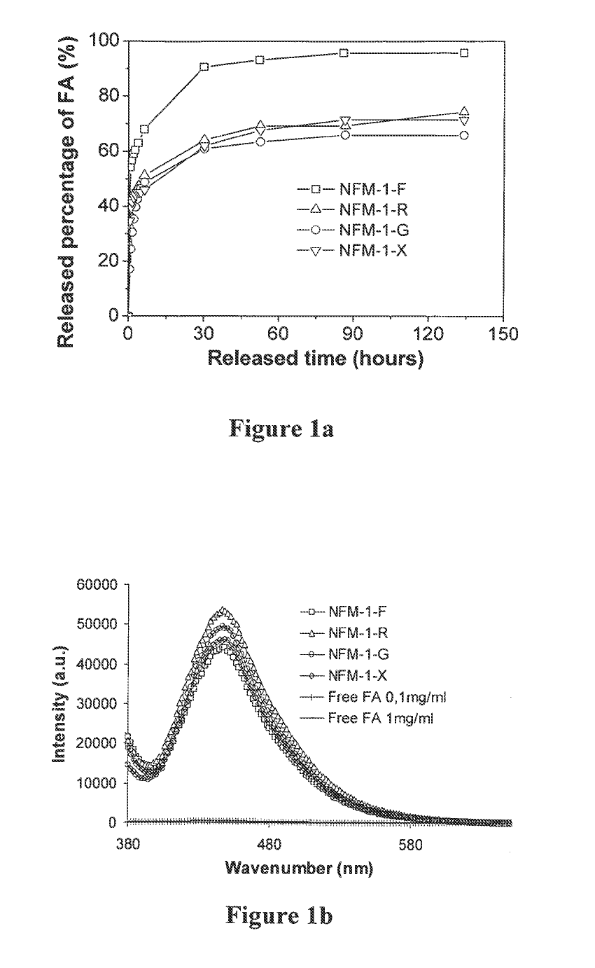

Single fluorescent folic acid materials (NFM-1) with enhanced fluorescent properties can be formed as described in WO/2009/068117. FIG. 1 shows the release properties as well as fluorescent properties of NFM-1 materials with different morphologies, namely gyroid, fiber, rod type and amorphous particles. Release folate stacks from mesoporous silica particles show considerably higher fluorescent intensity in comparison to free folate in solution. Fiber type morphologies show slightly higher enhancement in fluorescence than other morphologies presumably because of the quicker release process.

Multi-fluorescence materials are prepared by adding TCPP in the NFM-1 synthesis. These samples are denoted NFM-1P (x), here x is the ratio of folic acid to TCPP. Different amounts of TCPP were added in the synthesis as shown in the following Table 2.

TABLE 2

| | TCPP | | Folic acid | H₂O | APES | TEOS |
|---|---|---|---|---|---|---|
| | 790.8 | | 441.4 | 18 | 221.3 | 208.3 |
| NFM-1 | 0 | | | | | |
| NFM-1P(367) | 10 | mg | | | | |
| NFM-1P(16) | 52 | mg | 0.45 g | 32.5 g | 0.57 g | 1.6 g |
| NFM-1P(8) | 101 | mg | | | | |
| NFM-1P(5) | 158 | mg | | | | |

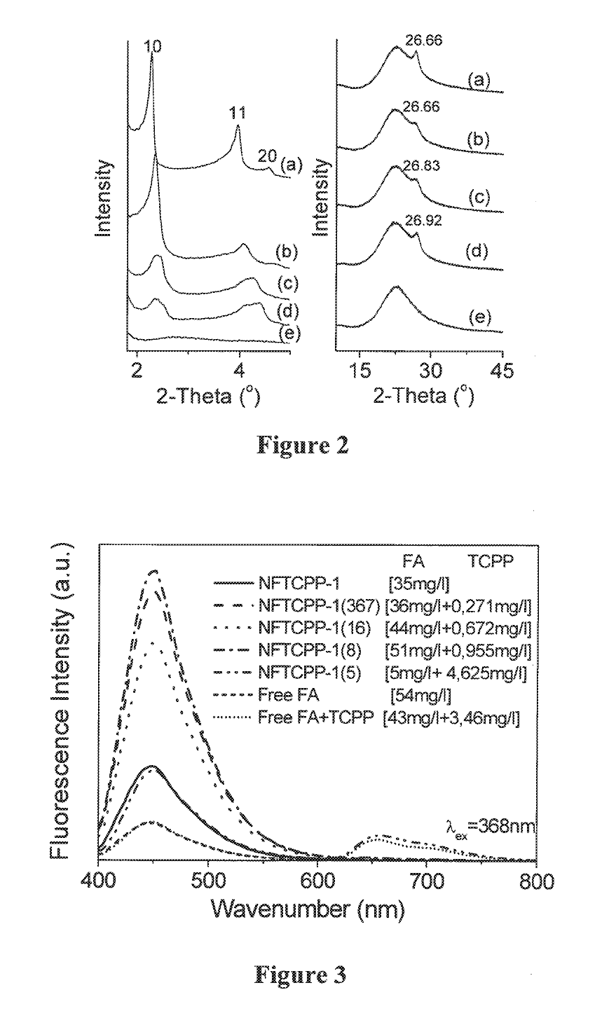

In order to get the textural characterization, Low-angle X-ray powder diffraction (XRD) patterns were performed on an X'Pert Pro diffractometer using Cu Kr radiation (λ=1.5418 Å) at 45 kV and 20 mA. FIG. 2 shows the low angle and high-angle X-ray diffraction (XRD) patterns of the samples with different loadings amount of TCPP showing that mesoscale order can be achieved even for ratios of folic acid to TCPP=8. It is clear from this data that samples have ordered mesoporous structure. However, the diffraction intensities decrease with the loading of TCPP, and the peaks position shifts to high angle. The peak at 26.66° denoted the π-π stack of folic acid also shifts a little as shown in the high-angle XRD, which prove TCPP are stacked among the tetramers of folic acid as shown in the following FIG. 9.

Further evidence proving the stacking of TCPP within folic tetramers is the fluorescence spectrum of the NFM-1P(x) samples as shown in FIG. 3. Here, NFM-1P(5) sample have no ordered structure as shown from low-angle XRD data. As for the ordered mesoporous materials, we can find the more TCPP, and the higher fluorescence intensity of folic acid, but the lower fluorescence intensity of TCPP. The addition of TCPP not only decreases the fluorescent self-quenching of two close tetramers of folic acid but also of TCPP molecules. The result of NFM-1P(5) also shows that if too much TCPP molecules are added, it will block the formation of ordered structure and promote the fluorescence self-quenching.

### Example 2

Multifunctional NFM-1 particles offering both fluorescent and therapeutic agents within the pore space of the mesoporous material are prepared by adding cisplatin in the NFM-1 synthesis. These samples are denoted NFCP-1 (x), here x is the ratio of folic acid to cisplatin. Different amounts of cisplatin can be added in the synthesis shown in the following Table 3 including ratios of up to 1:1 without loss of the hexagonal mesoscale order of the pore arrangement in the final product.

TABLE 3

| | Cisplatin(CP) | | NaCl | Folic acid | H₂O | APES | TEOS |
|---|---|---|---|---|---|---|---|
| | 300.01.00 | | 58.04.00 | 441.04.00 | 18 | 221.03.00 | 208.03.00 |
| NFCP-1 | 0 | | | | | | |
| NFCP-1(10) | 30.2 | mg | 0.29 g | 0.45 g | 32.5 g | 0.57 g | 1.6 g |
| NFCP-1(5) | 63 | mg | | | | | |
| NFCP-1(2) | 127 | mg | | | | | |

In order to get the textural characterization, Low-angle X-ray powder diffraction (XRD) patterns were performed on an X'Pert Pro diffractometer using Cu Kr radiation (λ=1.5418 Å) at 45 kV and 20 mA.

FIG. 3 shows the low angle and high-angle X-ray diffraction (XRD) patterns of the samples with different loading amount of cisplatin. All of the showed samples show two well resolved diffraction peaks. It is clear from this data that samples have ordered mesoporous structure, which means the cisplatin molecules are inserted in the stacks of folate. However, the diffraction intensities decrease with the loading amount due to the low shape matching between the cisplatin molecules and folate tetramers. The peak at 26.66° denoted the π-π stack of folic acid also shifts a little as shown in the high-angle XRD, which also prove cisplatin therapeutic agents are stacked among the tetramers of folic acid.

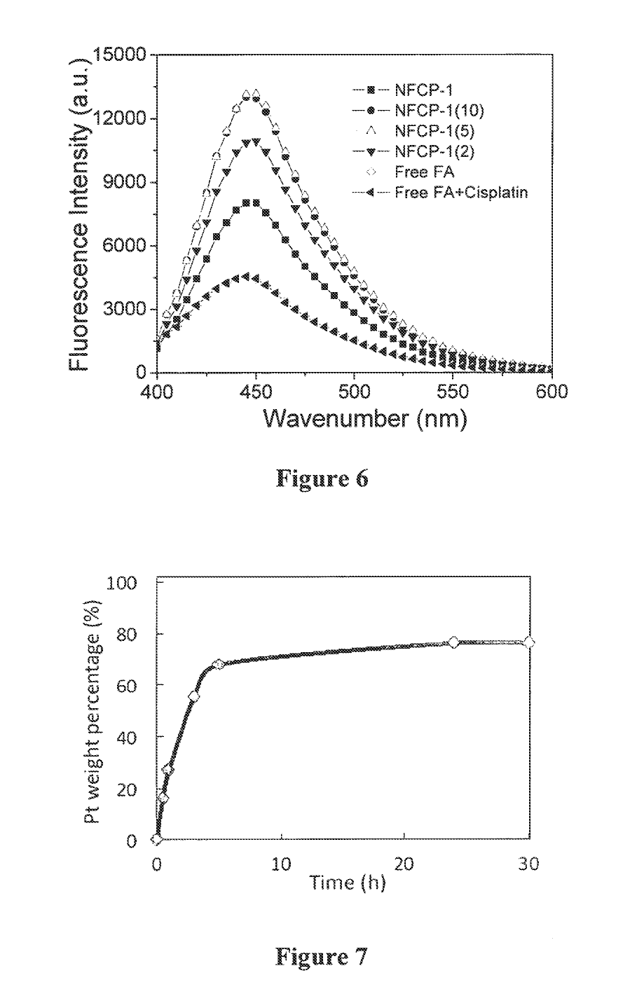

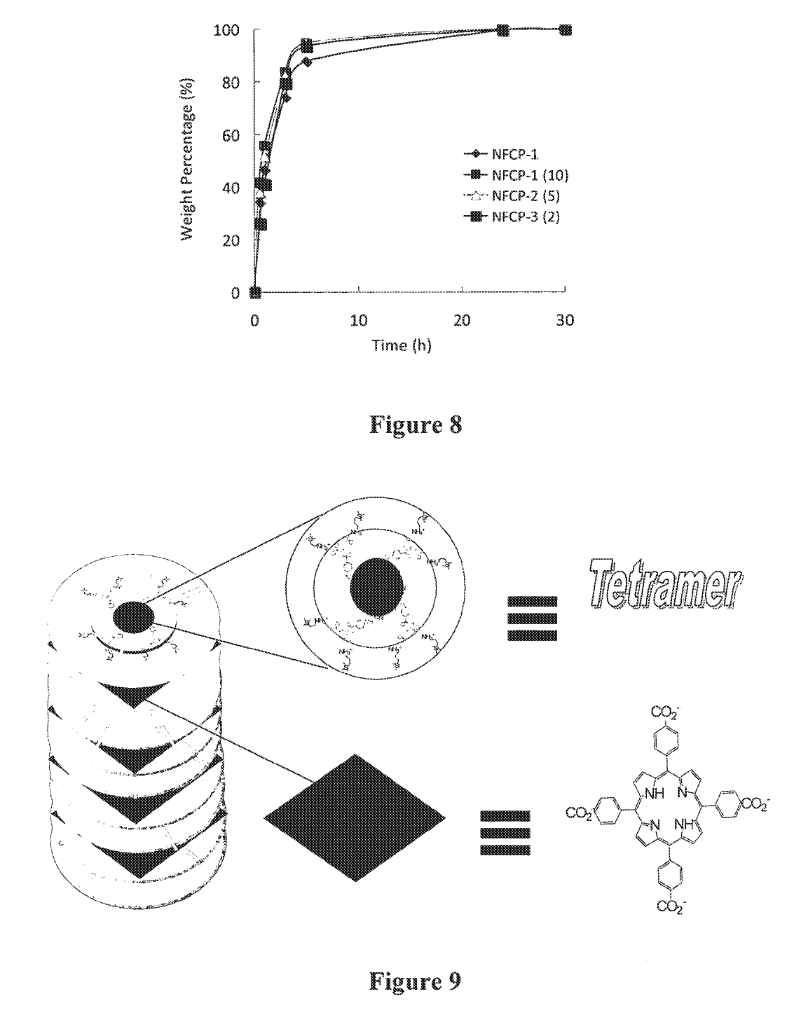

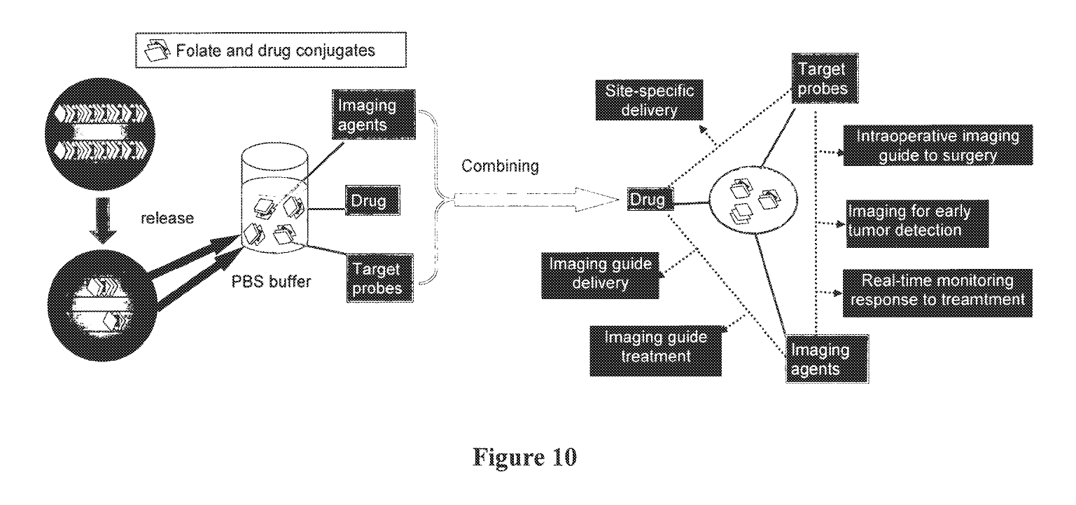

Further evidence proving the stacking of cisplatin within folic tetramers is the fluorescence spectrum of the NFCP-1(x) samples as shown in FIG. 6. The more cisplatin drugs, the lower fluorescence intensity of folic acid is as shown in FIG. 6. The effects of the therapeutic agents cisplatin on the fluorescence properties of folic acid are reversed comparing with the porphyrin. One possibility is the porphyrin could be the electron donor; however the metal in cisplatin could be the electron acceptor.

1. A method of releasing stacks of folic acid compounds and theranostic compounds into a solvent wherein said stacks are released into water, buffer or organic solvent from a theranostic porous or non-porous material comprising two or more compounds, wherein one of said compounds is folic acid and whereby at least one compound experiences an alteration in its fluorescent intensity due to its self-assembly via pi-pi or pi-sigma interactions with folic acid.

2. The method of releasing stacks of folic acid compounds and theranostic compounds according to claim 1, wherein said material possesses hexagonal mesoscale order.

3. The method of releasing stacks of folic acid compounds and theranostic compounds according to claim 1, wherein one of the compounds is a fluorophore or pharmaceutical therapeutic compound capable of via pi-pi or pi-sigma interactions with folic acid.

4. The method of releasing stacks of folic acid compounds and theranostic compounds according to claim 1, where the fluorescent molecule is a porphyrin.

5. The method of releasing stacks of folic acid compounds and theranostic compounds according to claim 1, where the compound other than folic acid is composed of a pharmaceutical therapeutic compound is cis-Pt.

6. The method of releasing stacks of folic acid compounds and theranostic compounds according to claim 1, wherein the porosity is described as a material possessing a pore volume between 0.01 and 0.9 cm³/g as measured by nitrogen adsorption isotherm.

7. The method of releasing stacks of folic acid compounds and theranostic compounds according to claim 1, wherein the porosity is described as a having a surface area between 10 and 1500 m²/g as measured by nitrogen adsorption isotherms.

8. The method of releasing stacks of folic acid compounds and theranostic compounds according to claim 1, wherein a first fluorophore is present in an amount between 1-40 wt % of the total material weight as measured by thermogravimetric analysis.

9. The method of releasing stacks of folic acid compounds and theranostic compounds according to claim 1, wherein a second fluorophores or pharmaceutical therapeutic compound is present in an amount between 1-39 wt % of the total material weight as measured by thermogravimetric analysis.

10. The method of releasing stacks of folic acid compounds and theranostic compounds according to claim 9, wherein the composition weight ratio of the first and second fluorophores is between 6-15.

11. The method of releasing stacks of folic acid compounds and theranostic compounds according to claim 1, which further comprises a third fluorophore and where the third fluorophore is a molecule of the group: Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, TRITC, X-Rhodamine, Lissamine Rhodamine B, Texas Red, Allophycocyanin (APC), APC-Cy7 conjugates.

12. A theranostic porous material as that described in claim 1, containing ordered hexagonal channels of diameter between 2-5 nm and a composition of at least 30 wt % of silicon oxide (silica).

13. The method of releasing stacks of folic acid compounds and theranostic compounds according to claim 1, containing tethered propyl amine groups spaced out at a distance not larger than 6 Å within the internal pore surface of the material.

14. The method of releasing stacks of folic acid compounds and theranostic compounds according to claim 1, wherein fluorescent enhancement is of 90% in intensity in comparison to the free fluorophores in solution.

15. The method of releasing stacks of folic acid compounds and theranostic compounds according to claim 1, wherein the fluorescent emission spectra has a peak maxima at between 350 and 375 nm.

16. The method of releasing stacks of folic acid compounds and theranostic compounds according to claim 1, wherein fluorescent enhancement occurs as a result of Forster resonance energy transfer within the internal channel space of the material.

17. The method of releasing stacks of folic acid compounds and theranostic compounds according to claim 1, wherein the fluorophores maintain their enhanced properties when released into aqueous and non-aqueous media.

18. The method of releasing stacks of folic acid compounds and theranostic compounds according to claim 17, wherein the fluorophores maintain their enhanced properties when released into said aqueous and non-aqueous media.

19. The method of releasing stacks of folic acid compounds and theranostic compounds according to claim 1, wherein the theranostic porous material carrier is for cellular targeting, cellular imaging, diagnosis and therapy containing at least 1 wt % of self-assembled folic acid.

20. The method of releasing stacks of folic acid compounds and theranostic compounds according to claim 19, wherein the targeting molecule is folic acid or folate derivative characterized by being capable of binding to folate receptors in cell membranes.

21. The method of releasing stacks of folic acid compounds and theranostic compounds according to claim 19, wherein the content of the therapeutic agent is between 0.01-39 wt % of the total material weight as measured by thermogravimetric analysis and is delivered.

22. The method of releasing stacks of folic acid compounds and theranostic compounds according to claim 1, wherein the theranostic porous material is capable of cellular targeting to tumor cells, and wherein the fluorescent material is folic acid, folic acid and a porphyrin or a folate derivative and porphyrin derivative; and the therapeutic agent are photosensitizers.

23. The method of releasing stacks of folic acid compounds and theranostic compounds according to claim 1, wherein the therapeutic agents are antimetabolites: dyhydrofolate reductase inhibitor; purine analogue; pyrimidine analogue; or Topoisomerase inhibitors; or crosslinkers in DNA; or Mitotic inhibitor; or enzyme inhibitors; or are receptor antagonists; or chemotherapeutical agents.

24. The method of releasing stacks of folic acid compounds and theranostic compounds according to claim 1, wherein said stacks are released into water, buffer or organic solvent at a temperature of 4° C. to 100° C.

25. The method of releasing stacks of folic acid compounds and theranostic compounds according to claim 1, wherein said stacks are released into water, buffer or organic solvent at a temperature of 25° C. to 40° C.
